(19) United States
(12) Patent Application Publication
(10) Pub. No.: US 2022/0336087 A1
(43) Pub. Date: Oct. 20, 2022

(54) METHODS AND SYSTEMS FOR SCHEDULING A PATIENT-SPECIFIC IMMUNOTHERAPY PROCEDURE

(71) Applicant: Kite Pharma, Inc., Santa Monica, CA (US)

(72) Inventor: Charles H. Wilke, Calabasas, CA (US)

(21) Appl. No.: 17/715,703

(22) Filed: Apr. 7, 2022

Related U.S. Application Data
(60) Provisional application No. 63/176,018, filed on Apr. 16, 2021.

(51) Int. Cl. G16H 40/20; G06Q 10/10

(52) U.S. Cl. CPC G16H 40/20; G06Q 10/1095; A61M 1/3496

(57) ABSTRACT

A method and system for scheduling a patient-specific immunotherapy procedure. The method includes receiving a request on a server computing device to schedule a leukapheresis appointment for an available date and time displayed on a calendar stored in a database that is in communication with the computing device. The request can be made with a client computing device that is in communication with the server computing device via a network. Furthermore, the calendar may be updated in real time by the server computing device to show any changes to the calendar. The server computing device may automatically generate a final product date to estimate when the final product of engineered cells from the patient will be available for infusion into the patient. A manufacturing processing status for the final product of engineered cells from the patient may be updated on the calendar.

### CROSS-REFERENCE TO RELATED APPLICATIONS

This application claims the benefit under 35 U.S.C. § 119(e) of U.S. Provisional Application No. 63/176,018, filed on Apr. 16, 1021 the entirety of which is incorporated herein by reference.

### TECHNICAL FIELD

This application relates generally to methods and apparatuses, including computer program products, for performing a patient-specific immunotherapy procedure with a dynamic scheduling calendar and chain-of-custody and chain-of-identity biological sample tracking.

### BACKGROUND

In recent years, advances in medical technology have led to the emerging use of immunotherapies to treat different types of illnesses and diseases, including various forms of cancer. Generally, immunotherapy is the treatment of disease by stimulating or suppressing an immune response. Often, modified versions of a patient's own biological material, such as immune cells, are reintroduced into the patient in order to initiate and/or supplement the immune response.

For example, engineered immune cells have been shown to possess desired qualities in therapeutic treatments, particularly in oncology. Two main types of engineered immune cells are those that contain chimeric antigen receptors (termed “CARs” or “CAR-Ts”) and T-cell receptors (“TCRs”). These engineered cells are engineered to endow them with antigen specificity while retaining or enhancing their ability to recognize and kill a target cell. Chimeric antigen receptors may comprise, for example, (i) an antigen-specific component (“antigen binding molecule”), (ii) an extracellular domain, (iii) one or more costimulatory domains, and (iv) one or more activating domains. Each domain may be heterogeneous, that is, comprised of sequences derived from (or corresponding to) different protein chains.

Because many patients that undergo immunotherapy are critically ill, a crucial factor in the efficacy of such immunotherapy procedures is the ability to provide the modified biological material to the patient as quickly as possible, so that the therapeutic benefits may be maximized. Also, because many types of immunotherapy are tailored for the specific patient (i.e., using the patient's own cells), it is important to ensure that the patient's biological material is accurately tracked throughout the immunotherapy process—from extraction of blood cells, to modification of those cells, and then to infusion of the cells back into the patient—to avoid delays in manufacturing, mislabeling of material, and misidentification of the patient. However, existing immunotherapy procedures generally lack a technical mechanism to track the patient's biological material automatically and to ensure that the biological material is tied to the specific patient's identity throughout the manufacturing process.

Furthermore, existing immunotherapy procedures, including calendaring procedures, are not dynamic Existing immunotherapy procedures have limited flexibility in scheduling a date for extraction, modification, and infusion of cells back into the patient. In addition, existing immunotherapy procedures do not provide simultaneous, real-time updates to multiple users involved in the immunotherapy process, including the patient, health professionals, patient case managers, members of the manufacturing facility where cells are modified, and system administrators.

### SUMMARY

Therefore, what is needed are methods and systems for performing a patient-specific immunotherapy procedure with chain-of-custody and chain-of-identity biological sample tracking. Furthermore, what is needed are methods and systems that are dynamic and allow flexibility in scheduling important dates in the patient-specific immunotherapy, along with providing real time updates to multiple users involved in the process, including patients, physicians or health care professionals, patient case managers, members of the manufacturing facility and system administrators. In addition, the techniques described herein provide the specific technical advantage over existing systems of providing a continuous and automatic chain of custody and chain of identity for a patient-specific biological sample during an immunotherapy procedure, to create a computerized information portal that interested parties—such as the patient, physician, manufacturer, and other medical personnel— may use to quickly understand and track the current phase of the immunotherapy procedure and the status of the patient's biological sample during the procedure. Such advanced tracking is an improvement over existing systems that do not have technological solutions for maintaining a chain of custody and chain of identity— resulting in delays during the manufacturing process which, for a patient dealing with a life-threatening illness, may be immeasurably severe.

Briefly, and in general terms, the present disclosure is directed to various embodiments of a method for scheduling a patient-specific immunotherapy procedure. The method includes receiving a request on a scheduling module or a server computing device to schedule a leukapheresis appointment for an available date and time displayed on a calendar stored in a database that is in communication with the scheduling module or server computing device. The request may be made with a client computing device that is in communication with the scheduling module via a network. Furthermore, the calendar may be updated in real time by the scheduling module to show any changes to the calendar. The updates to the calendar in real time may display available date and time slots for leukapheresis appointments and booked date and time slots for leukapheresis appointments. Furthermore, the calendar may include links in order to view specific patient information. The updated calendar may be viewed on multiple client computing devices connected to the scheduling module via the network.

In certain embodiment, requests may be made to the scheduling module to cancel or reschedule the leukapheresis appointment for another available date and time displayed on the calendar. If changes are made to the calendar, the scheduling module may update the calendar in real time so that all users of the system are aware of the changes made.

In one embodiment, the scheduling module may automatically generate a final product date to estimate when the final product of engineered cells from the patient will be available for infusion into the patient. A manufacturing processing status for the final product of engineered cells from the patient also may be updated on the calendar. These updates may be received and displayed by the system in real time.

In one embodiment, the method may also include automatically scheduling by the scheduling module an apheresis kit drop-off time before the scheduled leukapheresis appointment. The apheresis kit is used by the treating facility during the leukapheresis appointment. Furthermore, the scheduling module updates the calendar in real time to include the drop off time for the apheresis kit. The method may also automatically send an alert from the scheduling module to a remote facility or a manufacturing facility with the scheduled drop-off time and a desired location for the apheresis kit. This will alert the remote facility to deliver the apheresis kit at the scheduled drop-off time to the desired location.

In another embodiment, the method may include receiving a request on the scheduling module to schedule a courier pick-up time for cells collected from the patient during the scheduled leukapheresis appointment. In this way, the cells collected from the patient will be transported to the manufacturing facility with little delay.

In one embodiment, the method may include receiving a number of available leukapheresis appointments for each day on the calendar on the scheduling module. In this way, the manufacturing facility can manage its production of final products. This helps to decrease the time between when a patient undergoes leukapheresis and when the final product or engineered cells is infused into the patient.

Another embodiment of the disclosure discloses a system for scheduling a patient-specific immunotherapy procedure. The system may include a scheduling module that provides or generates a calendar with available dates and times for scheduling a leukapheresis appointment. The scheduling module may be a stand-alone module or it may be incorporated into a server device that has multiple functions. Also, a database may be in communication with the scheduling module to store the calendar and any updates to the calendar. There may also be a network in communication with the scheduling module and a plurality of client computing devices in communication with the scheduling module via the network. In the system of one embodiment, the client computing devices may send a request to the scheduling module to schedule or reserve an available leukapheresis appointment date and time for the patient. Also, the scheduling module may receive the request to reserve or schedule the leukapheresis appointment for the patient and update the calendar in real time that can be accessed by each of the plurality of client computing devices.

In one embodiment of the system, the scheduling module automatically generates a final product date based on the scheduled leukapheresis appointment date and time. The final product date may estimate when the final product of engineered cells from the patient will be available for infusion into the patient. The scheduling module may update the calendar with the final product date.

In certain embodiments of the system, one of the client computing devices may send manufacturing process updates for the final product of engineered cells from the patient to the server computing device via the network. The server computing device may update the manufacturing status on the system so that the treatment facility and other users will be made aware if there are any delays in manufacturing.

In one embodiment the scheduling module automatically schedules an apheresis kit drop-off time before the scheduled leukapheresis appointment and updates the calendar to include the drop off time for the apheresis kit. The scheduling module may automatically send an alert to a remote facility with the scheduled drop-off time and a desired location for the apheresis kit, wherein the remote facility may deliver the apheresis kit at the scheduled drop-off time to the desired location.

In another embodiment, the computing devices may send a request to the scheduling module via the network to schedule a courier pick-up time for cells collected from the patient during the scheduled leukapheresis appointment.

In one embodiment, the system may include receiving a number of available leukapheresis appointments for each day on the calendar on the computing device. In this way, the manufacturing facility can manage its production of final products. This helps to decrease the time between when a patient undergoes leukapheresis and when the final product or engineered cells is infused into the patient.

The present disclosure, in one embodiment, features a method of performing a patient-specific immunotherapy procedure. A computing device receives a cell order request to create transfected T cells for a patient. The computing device generates a patient-specific identifier associated with the cell order request, the patient-specific identifier comprising a patient identity element, a sales order identifier, and a cell order lot number. The computing device initiates a process to create transfected T cells for infusion into the patient's bloodstream, the process comprising: performing a leukapheresis procedure on a sample of the patient's blood to collect T cells from the sample, transferring the collected T cells to a container, labeling the container with the patient-specific identifier, transmitting the collected T cells to a manufacturing facility, creating transfected T cells from the collected T cells using a cell modification technique, receiving the transfected T cells from the manufacturing facility, and infusing the transfected T cells into the patient's bloodstream. The computing device records a tracking event for each step in the process, each tracking event including the patient-specific identifier. The tracking events comprise a chain of custody of the patient's T cells during the process.

In another embodiment, a method of tracking a cell order during an immunotherapy procedure is disclosed. A computing device receives a cell order request for creating transfected T cells for a patient. The computing device generates a patient-specific identifier associated with the cell order request, the patient-specific identifier comprising a patient identity element, a sales order identifier, and a cell order lot number. The computing device monitors a process to create transfected T cells for infusion into the patient's bloodstream, the process comprising: receiving indicia that a leukapheresis procedure has been performed on a sample of the patient's blood to collect T cells from the sample, receiving indicia that the collected T cells have been transferred to a container, receiving indicia that the container has been labeled with the patient-specific identifier, receiving indicia that the collected T cells have been transmitted to a manufacturing facility, receiving indicia that transfected T cells have been created from the collected T cells using a cell modification technique, receiving indicia that the transfected T cells have been received from the manufacturing facility, and receiving indicia that the transfected T cells have been infused into the patient's bloodstream. The computing device records a tracking event when indicia are received, each tracking event including the patient-specific identifier. The computing device maintains a chain of custody of the patient's T cells by storing the tracking events during the process.

The present disclosure features another embodiment of a method of performing a patient-specific immunotherapy procedure. A cell order request to create transfected T cells for a patient is received. An event tracking module executed on a processor generates a patient-specific identifier associated with the cell order request. A process to create transfected T cells for infusion into the patient's bloodstream is initiated, comprising: performing a leukapheresis procedure on a sample of the patient's blood to collect T cells from the sample, transferring the collected T cells to a container, labeling the container with the patient-specific identifier, transmitting the collected T cells to a manufacturing facility, creating transfected T cells from the collected T cells using a cell modification technique, receiving the transfected T cells from the manufacturing facility, and infusing the transfected T cells into the patient's bloodstream. The event tracking module receives, from a first client device located at the point of the leukapheresis procedure, a first tracking event that confirms the leukapheresis procedure and contains the patient-specific identifier. The event tracking module integrates the first tracking event in a data structure pertaining to the patient-specific identifier, where the data structure is stored in a database and the integrating step records a first timestamp with the first tracking event. The event tracking module receives, from a second client device located at the manufacturing facility, a second tracking event that confirms the receipt of the collected T cells at the manufacturing facility and contains the patient-specific identifier. The event tracking module integrates the second tracking event in the data structure pertaining to the patient-specific identifier, where the integrating step records a second timestamp with the second tracking event.

In one embodiment, a method of performing a patient-specific immunotherapy procedure is disclosed. A tracking module executed on a processor receives a cell order request to create transfected T cells for a patient. The tracking module generates a patient-specific identifier associated with the cell order request, the patient-specific identifier identifying a patient, and a cell order lot. A database generates a data record for tracking the cell order, the data record identified in the database according to the patient-specific identifier. The tracking module receives a first tracking event indicating that the collected T cells are ready for shipment to a manufacturing facility. The data record corresponding to the patient-specific identifier is updated in accordance with the first tracking event. The tracking module receives, based on the container having been received by the manufacturing facility, a second tracking event indicating that the collected T cells have been received by a manufacturing facility. The data record corresponding to the patient-specific identifier is updated in accordance with the second tracking event. The tracking module receives, based on the manufacturing facility having created transfected T cells from the collected T cells using a cell modification technique, a third tracking event indicating that the transfected T cells have been created. The data record corresponding to the patient-specific identifier is updated in accordance with the third tracking event. The tracking module receives, based on the transfected T cells having been received from the manufacturing facility, a fourth tracking event indicating that the transfected T cells have been received. The data record corresponding to the patient-specific identifier is updated in accordance with the fourth tracking event. The tracking module receives, based on the transfected T cells having been infused into the patient's bloodstream, a fifth tracking event indicating that the transfected T cells have been infused into the patient's bloodstream. The data record corresponding to the patient-specific identifier is updated in accordance with the fifth tracking event, where each of the first, second, third, fourth, and fifth tracking events contains the patient-specific identifier, a timestamp, and an event identifier, and where the data record corresponding to the patient-specific identifier stores, in an ordered sequence, the first, second, third, fourth, and fifth tracking events when the data record is updated in accordance with the respective events.

Other features of the methods and systems disclosed herein will become apparent from the following detailed description, taken in conjunction with the accompanying drawings, which illustrate by way of example, the features of the various embodiments.

### BRIEF DESCRIPTION OF THE DRAWINGS

The teachings claimed and/or described herein are further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1A is a block diagram of a system for performing a patient-specific immunotherapy procedure with chain-of-custody and chain-of-identity biological sample tracking.

FIG. 1B is a detailed block diagram of a system for performing a patient-specific immunotherapy procedure with chain-of-custody and chain-of-identity biological sample tracking.

FIG. 2 is a flow diagram of a computerized method of performing a patient-specific immunotherapy procedure with chain-of-custody and chain-of-identity biological sample tracking.

FIGS. 3A and 3B are exemplary screenshots generated by a user interface module to receive patient-specific information during a patient-specific immunotherapy procedure.

FIGS. 4A to 4D are exemplary screenshots of one embodiment for generating by a user interface module to receive confirmation of extraction and infusion sites, and to schedule an appointment, during a patient-specific immunotherapy procedure.

FIGS. 5A to 5G are exemplary screenshots of another embodiment for generating by a user interface module to register a patient for an immunotherapy procedure, schedule appointments, and make changes to the appointments with a real time calendaring system.

FIGS. 6A and 6B are exemplary screenshots generated by a user interface module to display a chain of custody for biological material during a patient-specific immunotherapy procedure.

### DETAILED DESCRIPTION

The present disclosure addresses the need for an improved immunotherapy procedure that allows for dynamic scheduling and improved chain-of-custody and chain-of-identity. The below disclosure describes the methods and systems in the context of performing a patient-specific immunotherapy procedure. It should be understood, however, that the disclosure can apply equally to any procedure or process requiring real time scheduling of events and tracking valuable cargo. In certain embodiments, the methods and systems may be dynamic by allowing flexibility in scheduling important dates in the patient-specific immunotherapy, along with providing real time updates to multiple involved in the process, including patients, physicians or health care professionals, patient case managers, members of the manufacturing facility and system administrators. By providing real time calendaring of procedures, manufacturing status, and estimation of the delivery date of a final product for an immunotherapy process helps improve the entire process so that the patient receives treatment as soon as possible.

It will be understood that descriptions herein are exemplary and explanatory only and are not restrictive of the invention as claimed. In this application, the use of the singular includes the plural unless specifically stated otherwise.

All documents, or portions of documents, cited in this application, including but not limited to patents, patent applications, articles, books, and treatises, are hereby expressly incorporated by reference in their entirety for any purpose. As utilized in accordance with the present disclosure, the following terms, unless otherwise indicated, shall be understood to have the following meanings:

In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

As used herein, “patient-specific immunotherapy procedure” means any procedure that uses molecular or cellular components of the immune system to target and/or destroy cancer, pathogenic, or other disease-causing cells. An immunotherapy procedure is “patient-specific” if it utilizes components of a patient's immune system to treat that patient's own cancer, pathology, or other disease.

As used herein, the terms “patient” and “subject” are used interchangeably and include human and non-human animals, as well as those with formally diagnosed disorders, those without formally recognized disorders, those receiving medical attention, those at risk of developing disorders, etc. In addition to humans, categories of animals within the scope of the present disclosure include, for example, agricultural animals, domestic animals, laboratory animals, etc. Some examples of agricultural animals include cows, pigs, horses, goats, etc. Some examples of domestic animals include dogs, cats, etc. Some examples of laboratory animals include rats, mice, rabbits, guinea pigs, etc.

The term “leukapheresis” refers to a specific form of apheresis which involves the selective separation and removal of leukocytes from a blood sample. During leukapheresis, the removed blood is passed through a cell separation device which separates nucleated white blood cells, including T cells, from red blood cells and plasma. The separated T cells may then be collected to be used in the cell modification techniques of the present disclosure. In certain embodiments, the red blood cells and plasma are returned to the individual as part of the separation process. In additional embodiments, the red blood cells and plasma are discarded or stored for further analysis.

As used herein, the terms “T cell” and “T lymphocyte” are interchangeable. T cells are a subset of lymphocytes defined by their development in the thymus and by heterodimeric receptors associated with the proteins of the CD3 complex. T cells of the present disclosure include, but are not limited to, naïve T cells, cytotoxic T cells, helper T cells, suppressor T cells, regulatory T cells, memory T cells, NKT cells, γδ cells, CD8αα cells, lymphokine activated cells, TCR-expressing cells, subtypes thereof, and any other cell type which may express chimeric receptor chain.

T cells may be engineered to possess specificity to one or more desired targets. For example, T cells may be transduced with DNA or other genetic material encoding an antigen binding molecule, such as one or more single chain variable fragment (“scFv”) of an antibody, in conjunction with one or more signaling molecules, and/or one or more activating domains, such as CD3 zeta. In addition to the CAR-T cells' ability to recognize and destroy the targeted cells, successful T cell therapy benefits from the CAR-T cells' ability to persist and maintain the ability to proliferate in response to antigen.

As used herein, the term “cell modification technique” includes, but is not limited to, transfection and transduction. The term “transfection” and grammatical variations thereof, refer to the introduction of foreign or exogenous DNA into a cell. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al., 1981, Gene 13:197. Transfection techniques include, but are not limited to, calcium phosphate-DNA co-precipitation, DEAE-dextran-mediated transfection, cationic lipid-mediated delivery, polybrene-mediated transfection, electroporation, sonoporation, microinjection, liposome fusion, lipofection (lipid transfection), polymer transfection, nanoparticles, polyplexes, receptor-mediated gene delivery, delivery mediated by polylysine, histone, chitosan, and peptides, protoplast fusion, retroviral infection, and biolistics (e.g. Gene Gun). The term “transduction” and grammatical variations thereof refer to the process whereby foreign DNA is introduced into a cell via viral vector. See Jones et al., (1998). Genetics: principles and analysis. Boston: Jones & Bartlett Publ.

As used herein, the term “infuse” and grammatical variations thereof mean to introduce a solution into a body through a blood vessel. An infusion of the present disclosure includes, but is not limited to, therapeutic introduction of a fluid other than whole blood into a blood vessel. For example, transfected T cells of the present disclosure may be infused into a patient's bloodstream, for example, intramuscularly, intravenously, intraarterially, intraperitoneally, or subcutaneously.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell therapies. T cell therapy may include adoptive T cell therapy, tumor-infiltrating lymphocyte (TIL) immunotherapy, autologous cell therapy, engineered autologous cell therapy (eACT), and allogeneic T cell transplantation. However, one of skill in the art would recognize that the conditioning methods disclosed herein would enhance the effectiveness of any transplanted T cell therapy. Examples of T cell therapies are described in U.S. Patent Publication Nos. 2014/0154228 and 2002/0006409, U.S. Pat. No. 5,728,388, and International Publication No. WO 2008/081035.

The T cells of the immunotherapy may come from a variety of sources. For example, T cells may be differentiated in vitro from a hematopoietic stem cell population, or T cells may be obtained from a subject. T cells may be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of known techniques, such as FICOLL™ separation and/or apheresis. Additional methods of isolating T cells for a T cell therapy are disclosed in U.S. Patent Publication No. 2013/0287748, which is herein incorporated by references in its entirety.

It will be appreciated that chimeric antigen receptors (CARs or CAR-Ts) are, and T cell receptors (TCRs) may, be genetically engineered receptors. These engineered receptors may be readily inserted into and expressed by immune cells, including T cells in accordance with techniques known in the art. With a CAR, a single receptor may be programmed to both recognize a specific antigen and, when bound to that antigen, activate the immune cell to attack and destroy the cell bearing that antigen. When these antigens exist on tumor cells, an immune cell that expresses the CAR may target and kill the tumor cell.

CARs may be engineered to bind to an antigen (such as a cell-surface antigen) by incorporating an antigen binding molecule that interacts with that targeted antigen. An “antigen binding molecule” as used herein means any protein that binds a specified target molecule. Antigen binding molecules include, but are not limited to antibodies and binding parts thereof, such as immunologically functional fragments. Peptibodies (i.e., Fc fusion molecules comprising peptide binding domains) are another example of suitable antigen binding molecules.

Preferably, target molecules may include, but are not limited to, blood borne cancer-associated antigens. Non-limiting examples of blood borne cancer-associated antigens include antigens associated with one or more cancers selected from the group consisting of acute myeloid leukemia (AML), chronic myelogenous leukemia (CML), chronic myelomonocytic leukemia (CMML), juvenile myelomonocytic leukemia, atypical chronic myeloid leukemia, acute promyelocytic leukemia (APL), acute monoblastic leukemia, acute erythroid leukemia, acute megakaryoblastic leukemia, lymphoblastic leukemia, B-lineage acute lymphoblastic leukemia, B-cell chronic lymphocytic leukemia, B-cell non-Hodgkin's lymphoma, myelodysplastic syndrome (MDS), myeloproliferative disorder, myeloid neoplasm, myeloid sarcoma), and Blastic Plasmacytoid Dendritic Cell Neoplasm (BPDCN).

In some embodiments, the antigen is selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD19, CD20, CD22, CD23, CD24, CD25, CD30, CD33, CD34, CD4, CD40, CD44, CD56, CD8, CLL-1, c-Met, CMV-specific antigen, CSPG4, CTLA-4, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGF1)-1, intestinal carboxyl esterase, kappa chain, LAGA-1a, lambda chain, Lassa Virus-specific antigen, lectin-reactive AFP, lineage-specific or tissue specific antigen such as CD3, MAGE, MAGE-A1 major histocompatibility complex (MHC) molecule, major histocompatibility complex (MHC) molecule presenting a tumor-specific peptide epitope, M-CSF, melanoma-associated antigen, mesothelin, mesothelin, MN-CA IX, MUC-1, mut hsp72, mutated p53, mutated p53, mutated ras, neutrophil elastase, NKG2D, Nkp30, NY-ESO-1, p53, PAP, prostase, prostase specific antigen (PSA), prostate carcinoma tumor antigen-1 (PCTA-1), prostate-specific antigen, prostein, PSMA, RAGE-1, ROR1, RU1, RU2 (AS), surface adhesion molecule, surviving and telomerase, TAG-72, the extra domain A (EDA) and extra domain B (EDB) of fibronectin and the A1 domain of tenascin-C (TnC A1), thyroglobulin, tumor stromal antigens, vascular endothelial growth factor receptor-2 (VEGFR2), virus-specific surface antigen such as an HIV-specific antigen (such as HIV gp120), as well as any derivate or variant of these surface markers.

In some embodiments, target molecules may include viral infection-associated antigens. Viral infections of the present disclosure may be caused by any virus, including, for example, HIV. This list of possible target molecules is not intended to be exclusive.

The TCRs of the present disclosure may bind to, for example, a tumor-associated antigen. As used herein, “tumor-associated antigen” refers to any antigen that is associated with one or more cancers selected from the group consisting of: adrenocortical carcinoma, anal cancer, bladder cancer, bone cancer, brain cancer, breast cancer, carcinoid cancer, carcinoma, cervical cancer, colon cancer, endometrial cancer, esophageal cancer, extrahepatic bile duct cancer, extracranial germ cell cancer, eye cancer, gallbladder cancer, gastric cancer, germ cell tumor, gestational trophoblastic tumor, head and neck cancer, hypopharyngeal cancer, islet cell carcinoma, kidney cancer, large intestine cancer, laryngeal cancer, leukemia, lip and oral cavity cancer, liver cancer, lung cancer, lymphoma, malignant mesothelioma, Merkel cell carcinoma, mycosis fungoides, myelodysplastic syndrome, myeloproliferative disorders, nasopharyngeal cancer, neuroblastoma, oral cancer, oropharyngeal cancer, osteosarcoma, ovarian epithelial cancer, ovarian germ cell cancer, pancreatic cancer, paranasal sinus and nasal cavity cancer, parathyroid cancer, penile cancer, pituitary cancer, plasma cell neoplasm, prostate cancer, rhabdomyosarcoma, rectal cancer, renal cell cancer, transitional cell cancer of the renal pelvis and ureter, salivary gland cancer, Sezary syndrome, skin cancers, small intestine cancer, soft tissue sarcoma, stomach cancer, testicular cancer, thymoma, thyroid cancer, urethral cancer, uterine cancer, vaginal cancer, vulvar cancer, and Wilms' tumor.

In certain embodiments, the present disclosure may be suitable for target molecule to hematologic cancer. In some embodiments, the cancer is of the white blood cells. In other embodiments, the cancer is of the plasma cells. In some embodiments, the cancer is leukemia, lymphoma, or myeloma. In certain embodiments, the cancer is acute lymphoblastic leukemia (ALL) (including non T cell ALL), acute lymphoid leukemia (ALL), and hemophagocytic lymphohistocytosis (HLH)), B cell prolymphocytic leukemia, B-cell acute lymphoid leukemia (“BALL”), blastic plasmacytoid dendritic cell neoplasm, Burkitt's lymphoma, chronic lymphocytic leukemia (CLL), chronic myelogenous leukemia (CML), chronic myeloid leukemia (CML), chronic or acute granulomatous disease, chronic or acute leukemia, diffuse large B cell lymphoma, diffuse large B cell lymphoma (DLBCL), follicular lymphoma, follicular lymphoma (FL), hairy cell leukemia, hemophagocytic syndrome (Macrophage Activating Syndrome (MAS), Hodgkin's Disease, large cell granuloma, leukocyte adhesion deficiency, malignant lymphoproliferative conditions, MALT lymphoma, mantle cell lymphoma, Marginal zone lymphoma, monoclonal gammapathy of undetermined significance (MGUS), multiple myeloma, myelodysplasia and myelodysplastic syndrome (MDS), myeloid diseases including but not limited to acute myeloid leukemia (AML), non-Hodgkin's lymphoma (NHL), plasma cell proliferative disorders (e.g., asymptomatic myeloma (smoldering multiple myeloma or indolent myeloma), plasmablastic lymphoma, plasmacytoid dendritic cell neoplasm, plasmacytomas (e.g., plasma cell dyscrasia; solitary myeloma; solitary plasmacytoma; extramedullary plasmacytoma; and multiple plasmacytoma), POEMS syndrome (Crow-Fukase syndrome; Takatsuki disease; PEP syndrome), primary mediastinal large B cell lymphoma (PMBCL), small cell- or a large cell-follicular lymphoma, splenic marginal zone lymphoma (SMZL), systemic amyloid light chain amyloidosis, T-cell acute lymphoid leukemia (TALL), T-cell lymphoma, transformed follicular lymphoma, Waldenstrom macroglobulinemia, or a combination thereof.

The TCRs of the present disclosure may also bind to a viral infection-associated antigen. Viral infection-associated antigens include antigens associated with any viral infection, including, for example, viral infection caused by HIV.

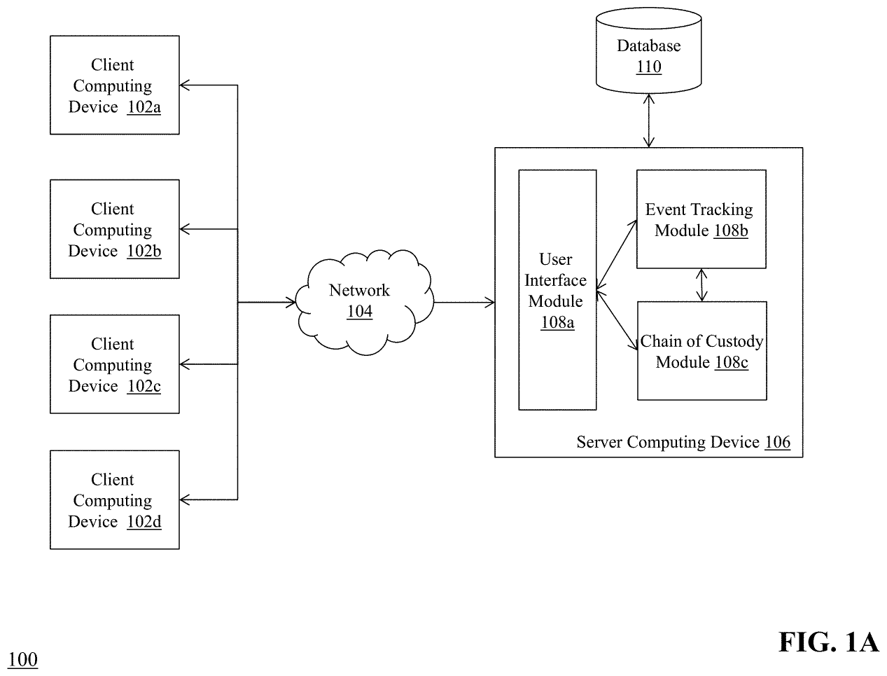

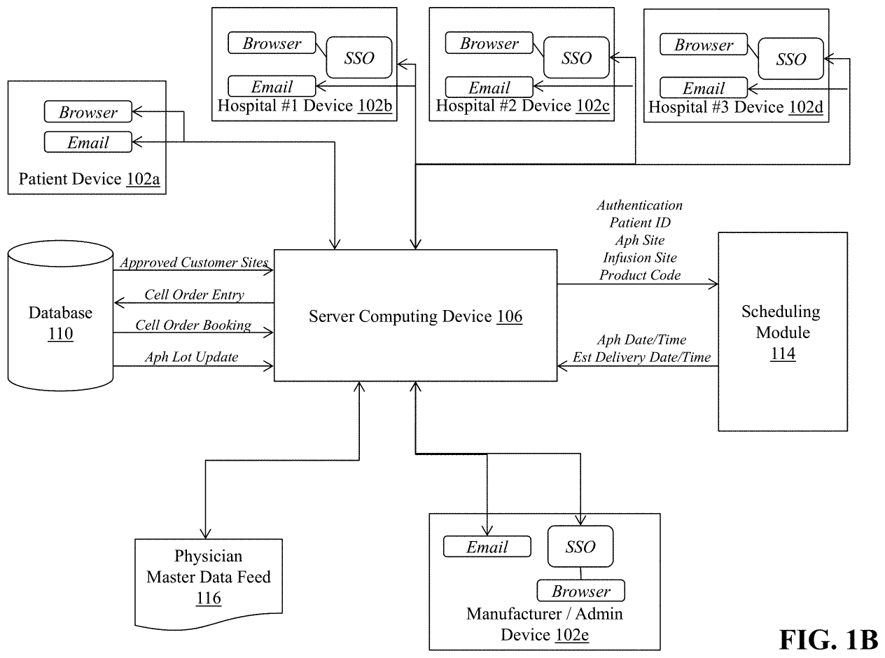

FIG. 1A is a block diagram of a system 100 for performing a patient-specific immunotherapy procedure with chain-of-custody and chain-of-identity biological sample tracking. The system of FIG. 1A includes a plurality of client computing devices 102a-102d, a communications network 104, a server computing device 106 with a user interface module 108a, an event tracking module 108b, and a chain of custody module 108c, and a database 110.

The client computing devices 102a-102d are connected to the communications network 104 in order to communicate with the server computing device 106 to provide input and receive output relating to the process of performing a patient-specific immunotherapy procedure with chain-of-custody and chain-of-identity biological sample tracking as described herein. In some embodiments, each client computing device 102a-102d may be coupled to a respective display device for, e.g., providing a detailed graphical user interface (GUI) that receives input for and presents output resulting from the methods and systems described herein. For example, the client computing device 102a-102d may connect to the user interface module 108a of server computing device 106, which provides, e.g., a web-based portal for users of the client computing devices 102a-102c to access functionality associated with the methods described herein.

Exemplary client devices 102a-102d include but are not limited to desktop computers, laptop computers, tablets, mobile devices, smartphones, and internet appliances. It should be appreciated that other types of computing devices that are capable of connecting to the components of the system of FIG. 1A may be used without departing from the scope of disclosure. It also should be appreciated that each of the client computing devices 102a-102d may be associated with a different user type—for example, client computing device 102a may be associated with a patient accessing the system of FIG. 1A to generate a user profile and receive updates on a patient-specific immunotherapy procedure; client computing device 102b may be associated with a physician who is treating the patient and who accesses the system of FIG. 1A to initiate an immunotherapy procedure for the patient; client computing device 102c may be associated with a hospital or other facility that is administering the immunotherapy procedure to the patient; and client computing device 102d may be associated with a manufacturing facility that is creating patient-specific immunotherapy product (as will be described herein) for use in the immunotherapy procedure.

The communications network 104 enables the other components of the system 100 to communicate with each other in order to conduct the process of performing a patient-specific immunotherapy procedure with chain-of-custody and chain-of-identity biological sample tracking as described herein. The network 104 may be a local network, such as a LAN, or a wide area network, such as the Internet and/or a cellular network. In some embodiments, the network 104 is comprised of several discrete networks and/or sub-networks (e.g., cellular to Internet) that enable the components of the system of FIG. 1A to communicate with each other.

The server computing device 106 is a combination of hardware and software modules that includes specialized hardware and/or software modules that execute on a processor and interact with memory modules of the server computing device 106 to perform functions for performing a patient-specific immunotherapy procedure with chain-of-custody and chain-of-identity biological sample tracking as described herein. The server computing device 106 includes a user interface module 108a, an event tracking module 108b, and a chain of custody module 108c (as mentioned above) that execute on and/or interact with the processor of the server computing device 106.

In some embodiments, the user interface module 108a, the event tracking module 108b, and the chain of custody module 108c are specialized sets of computer software instructions programmed onto one or more dedicated processors in the server computing device 106 and may include specifically-designated memory locations and/or registers for executing the specialized computer software instructions. Although the modules 108a-108c are shown in FIG. 1A as executing within the same server computing device 106, in some embodiments the functionality of the modules 108a-108c may be distributed among a plurality of server computing devices. As shown in FIG. 1A, the server computing device 106 enables the modules 108a-108c to communicate with each other in order to exchange data for the purposes of performing the described functions. It should be appreciated that any number of computing devices, arranged in a variety of architectures, resources, and configurations (e.g., cluster computing, virtual computing, cloud computing) may be used without departing from the scope of the disclosure. The exemplary functionality of the modules 108a-108c is described in detail below.

The database 110 is a computing device (or in some embodiments, a set of computing devices) coupled to the server computing device 106 and is configured to receive, generate, and store specific segments of data relating to the process of performing a patient-specific immunotherapy procedure with chain-of-custody and chain-of-identity biological sample tracking as described herein. In some embodiments, all or a portion of the database 110 may be integrated with the server computing device 106 or be located on a separate computing device or devices. The database 110 may comprise one or more databases configured to store portions of data used by the other components of the system of FIG. 1A, as will be described in greater detail below. In some embodiments, the database 110 comprises an enterprise business suite, such as Oracle E-Business Suite (EBS), that includes various modules that enable a spectrum of functionality to support the methods and systems described herein—including logistics, supply chain, transportation, CRM, and other types of modules.

FIG. 1B is a detailed block diagram of the system of FIG. 1A for performing a patient-specific immunotherapy procedure with chain-of-custody and chain-of-identity biological sample tracking. As shown in FIG. 1B, the server computing device 106 is the central component in the overall hardware architecture, interfacing with client computing devices 102a-102e and database 110, and also interfacing with a scheduling module 114 and a physician master data feed 116. In some embodiments, the server computing device 106 and the corresponding modules 108a-108c leverage the Salesforce platform, available from Salesforce.com, Inc. of San Francisco, Calif., to integrate certain of the functions described herein. The client computing devices 102a-102e communicate with the server computing device 106 to perform patient enrollment in the immunotherapy procedure and to monitor the chain-of-custody and chain-of-identity tracking (e.g., via browser-based user interfaces) as described herein. In one embodiment, the scheduling module 114 may be incorporated into the server computing device 106.

For example, client computing device 102a may be associated with the patient undergoing the immunotherapy procedure and may include browser software and email software to enable the patient to both monitor the tracking and to electronically sign documents required to participate in the immunotherapy procedure (e.g., via DocuSign or other similar technology). Similarly, the client computing devices 102b-102d may be located at different hospitals where a treating physician may enroll a patient in the immunotherapy procedure, place a cell order with the system, and monitor the chain-of-custody and chain-of identity tracking using the browser software. The client computing devices 102b-102d also include a single-sign-on (SSO) module that enables the devices to authenticate to the server computing device 106 (e.g., using SAML 2.0 supported SSO or a specific username/password for the server). The client computing device 102e may be located at an administration or manufacturing site to enable an administrator of the server computing device 106 to communicate with the server, receive communications such as emails from other participants in the system, and monitor the chain-of-custody and chain-of identity tracking using the browser software.

As described above, the database 110 may comprise an enterprise business suite that manages the data for the server computing device 106 and includes modules to enable chain-of-custody and chain-of-identity tracking and logistics for the biological sample. For example, the database 110 may transmit approved customer sites to the server 106 upon request, receive cell order entry data from the server 106, and provide cell order booking and apheresis lot update information to the server 106.

The scheduling module 114 may be integrated into the server computing device 106 or reside on a separate computing device. The scheduling module 114 may authenticate to and communicate with the server computing device 106 to receive certain information about the cell order and immunotherapy procedure (e.g., patient ID, apheresis site, infusion site, and product code) and provide calendaring and scheduling functionality to the server 106 (e.g., enabling a treating physician to select an apheresis date/time and provide an estimated delivery date/time for the biological sample once it has gone through the manufacturing process). Also, the server computing device 106 may communicate with a physician master data feed 116 (e.g., provided using the Veeva™ CRM platform integrated with the Heroku™ application) to receive certain information about treating physicians.

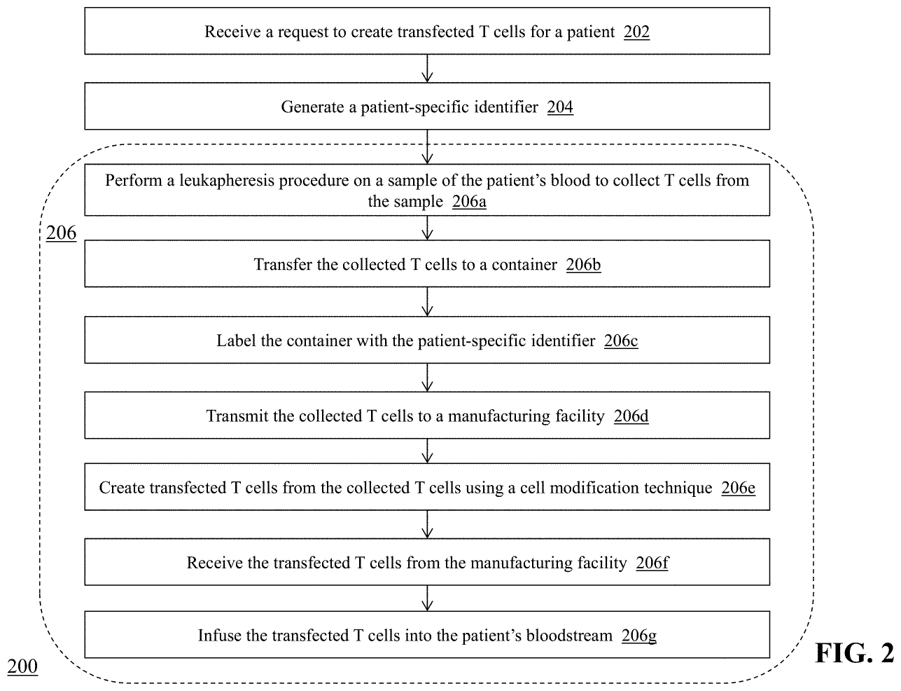

FIG. 2 is a flow diagram of a computerized method 200 of performing a patient-specific immunotherapy procedure with chain-of-custody and chain-of-identity biological sample tracking, using the system of FIG. 1A and/or the system of FIG. 1B.

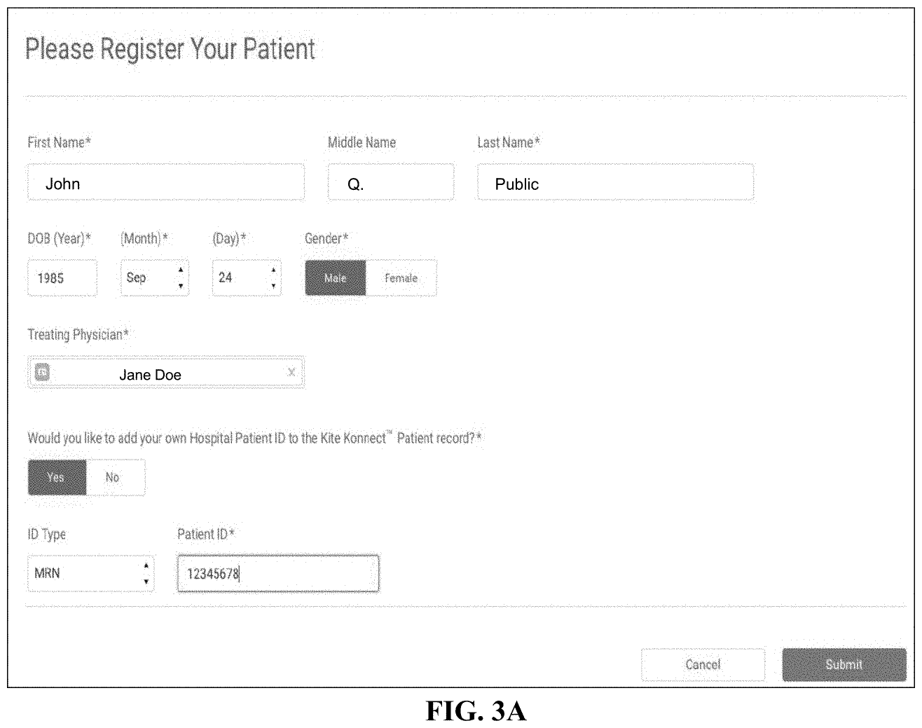

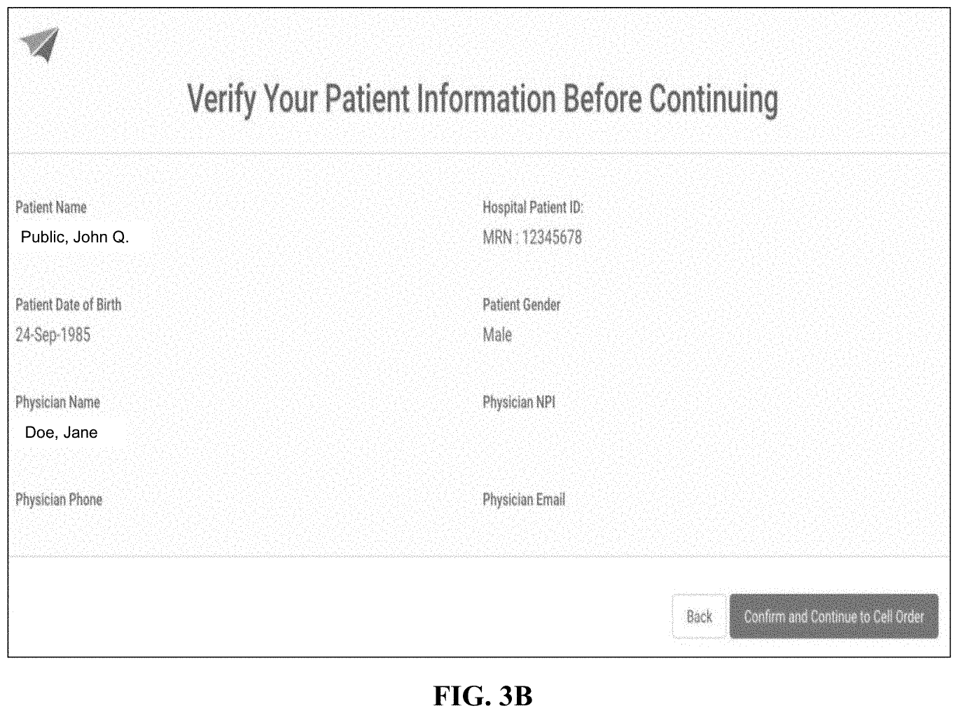

With reference to FIG. 1, to initiate the patient-specific immunotherapy procedure described herein, a physician or other medical personnel at client computing device 102a accesses the user interface module 108a of server computing device 106 (e.g., via a web portal, web site, or other similar platform). The user interface module 108a generates user interface screens and/or elements for presentation to the physician on the client computing device 102a, in order for the physician to enroll the patient and initiate the patient-specific immunotherapy procedure. The user interface module 108a may generate UI screens to enable the physician to enter the patient's identifying information (e.g., full name, date of birth), demographics (e.g., gender), and healthcare provider information (e.g., physician name, hospital name) The user interface module 108a may also provide a UI element for entry of a healthcare-provider-specific or hospital-specific user identifier (e.g., medical record number, hospital patient ID). FIGS. 3A and 3B are exemplary screenshots generated by the user interface module 108a that enable enrollment of new patients into the system; FIG. 3A depicts the patient enrollment data entry screen, and FIG. 3B depicts a patent information review and confirmation screen.

Referring to FIG. 2, in one embodiment, the client computing device 102a generates a request to create transfected T cells for a patient, and the server computing device 106 receives (202) the request. As described above, the physician at client computing device 102a interacts with the user interface module 108a to enroll the patient by providing the necessary patient information. Once the user interface module 108a receives confirmation from the client computing device 102a that the patient information has been fully entered and is accurate, the user interface module 108a stores the data in database 110. The user interface module 108a also generates (204) a patient-specific identifier that will be used as part of the sample tracking and chain-of-custody/chain-of-identity process described below. In one embodiment, the patient-specific identifier includes a patient identity element (e.g., a patient ID number), a sales order identifier, and a cell order lot number. For example, the user interface module 108a may generate the patient-specific identifier by mapping the patient identity element, sales order number, and cell order lot number into a database table that is indexed with an identifier (e.g., a nine-digit numeric code) that uniquely identifies the patient, sales order, and cell lot combination.

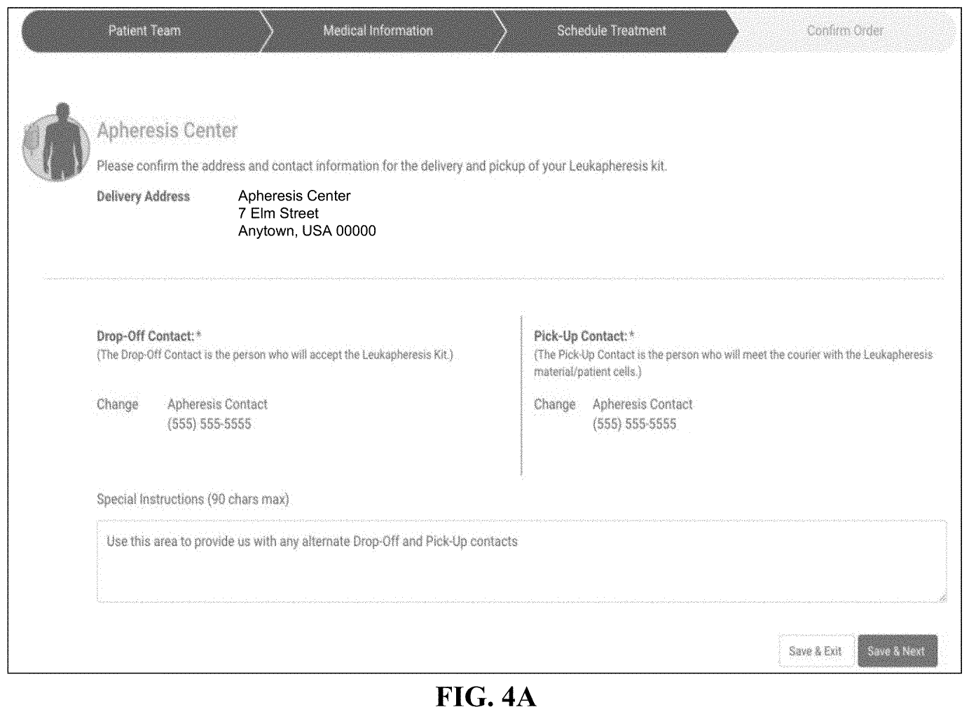

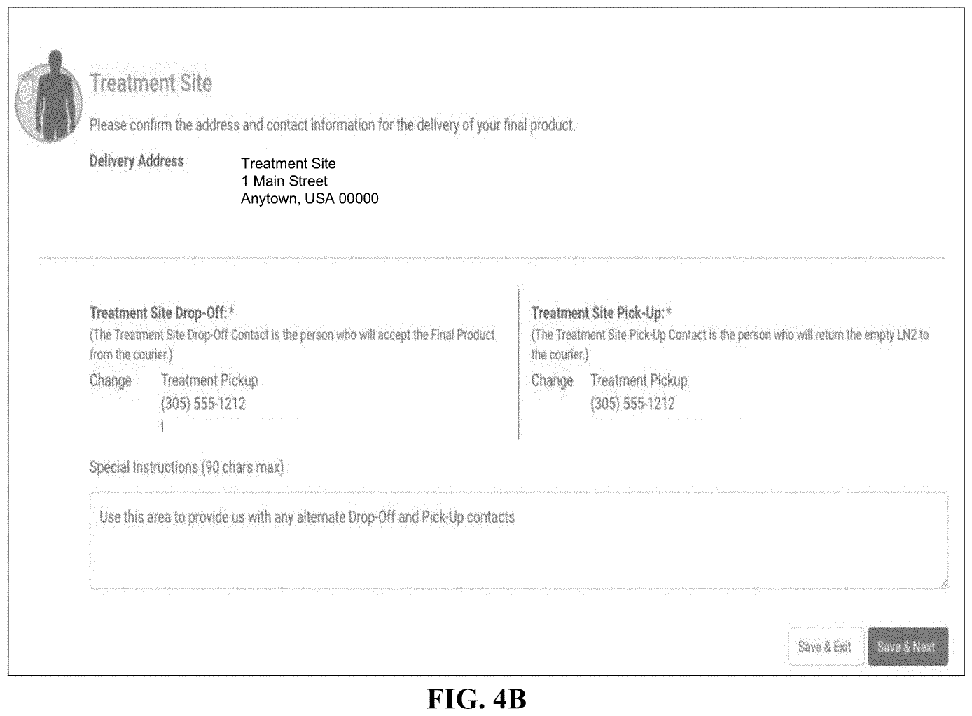

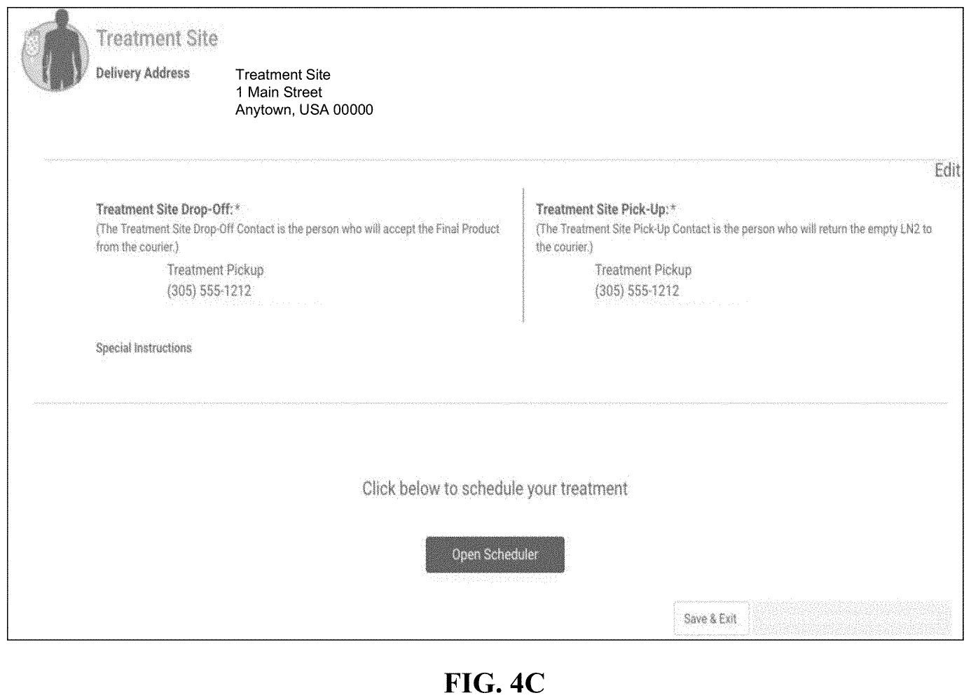

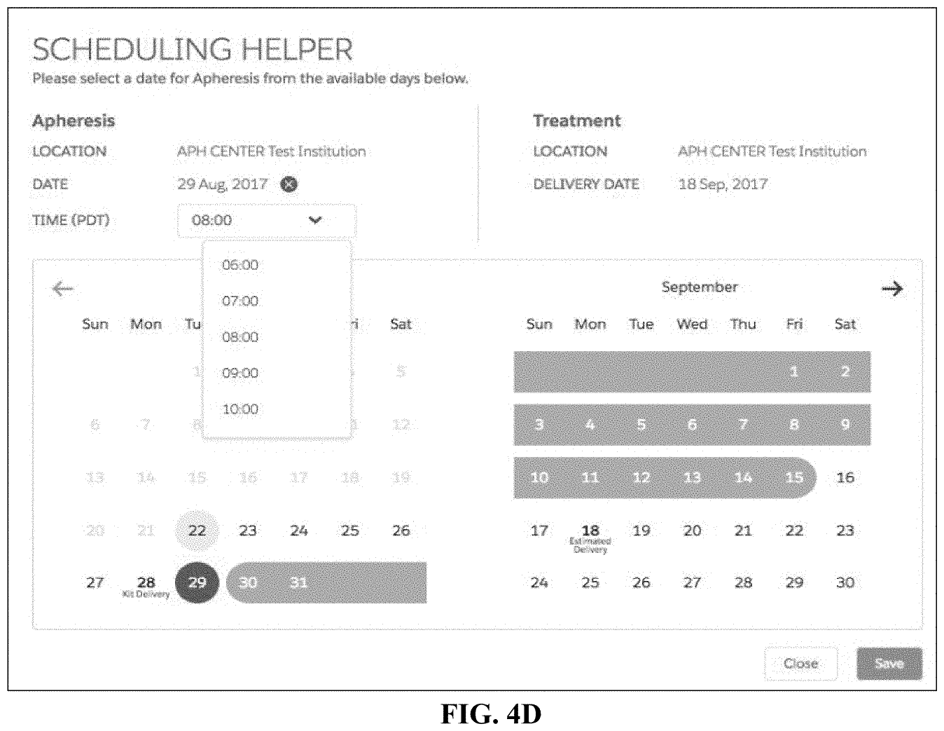

Next, the physician at client computing device 102a interacts with the user interface module 108a to schedule an appointment to obtain the biological material from the patient and, due to the time sensitivity of providing the altered biological material back to the patient quickly, confirming that the manufacturing facility has availability to process the biological material shortly after the material is obtained. The user interface module 108a requests confirmation of the material extraction site (e.g., site name, address, contact information) for drop-off of an extraction kit (e.g., leukapheresis kit) and confirmation of the altered material delivery and treatment site (e.g., site name, address, contact information) for delivery of the material (e.g., transfected T cells) from the manufacturing facility. FIGS. 4A-4D are exemplary screenshots generated by the user interface module 108a that enable confirmation of these sites and scheduling of the appointment; FIG. 4A depicts the drop-off site confirmation screen, FIG. 4B depicts the material delivery site confirmation screen; FIG. 4C depicts the screen to open the appointment scheduler; and FIG. 4D depicts the appointment scheduler. In some embodiments, the user interface module 108a communicates with a remote computing device of the manufacturing facility, in conjunction with the database 110, to coordinate scheduling of the biological material modification to ensure the most efficient processing schedule so that the modified material is returned quickly back to the patient.

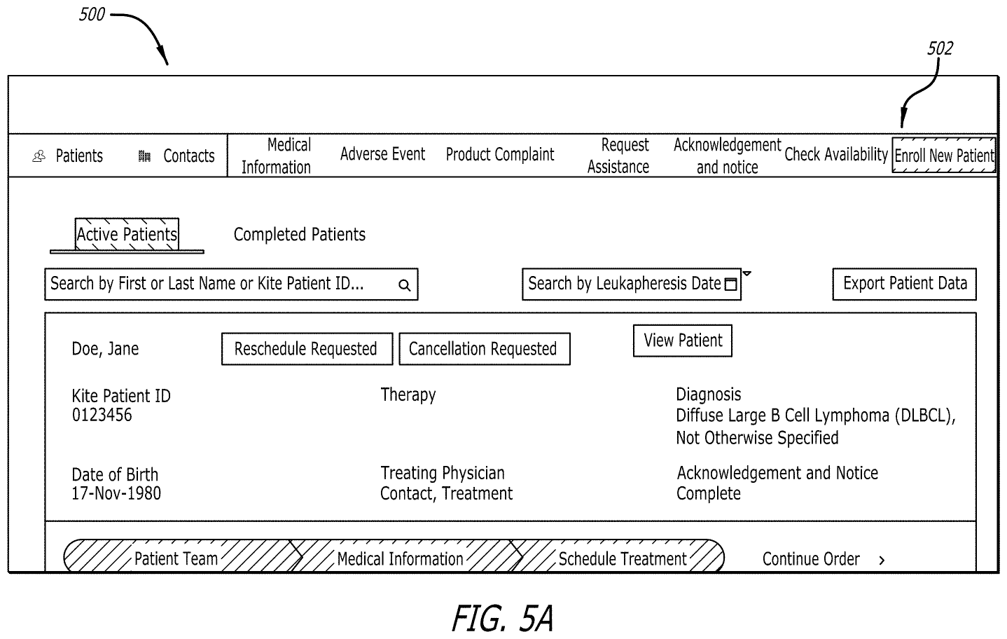

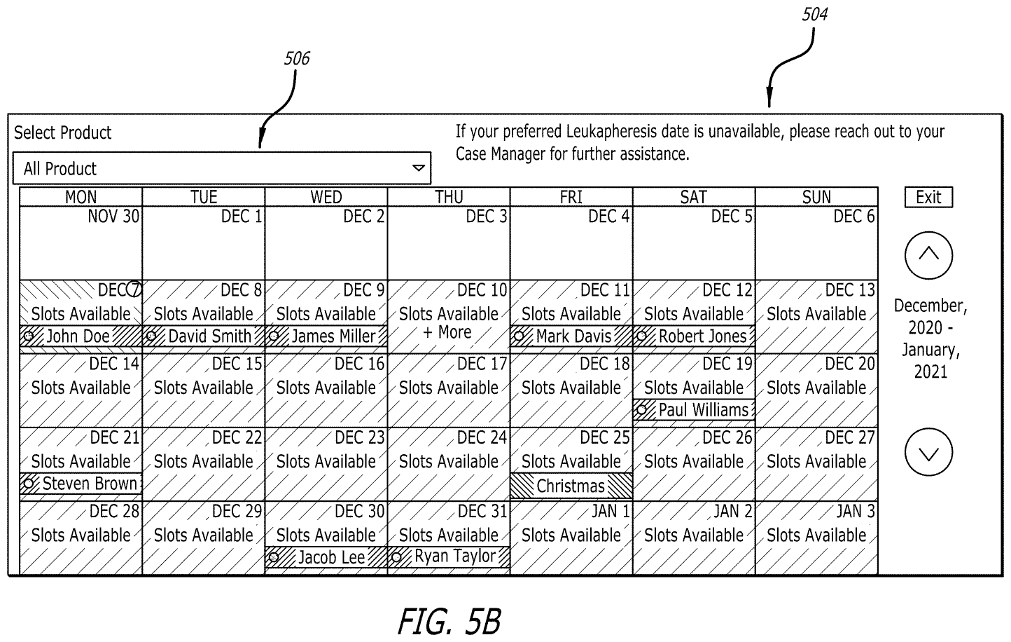

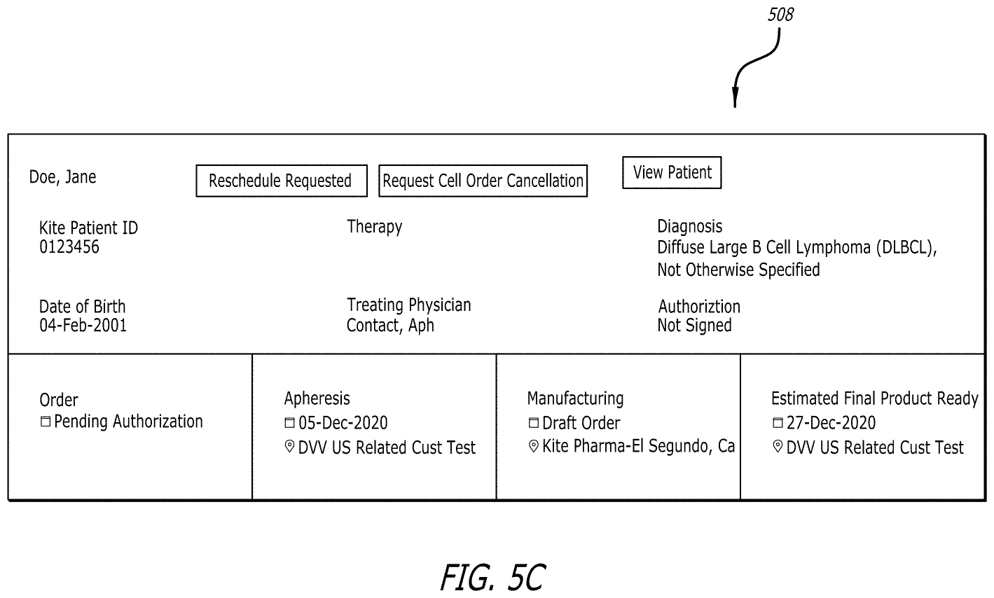

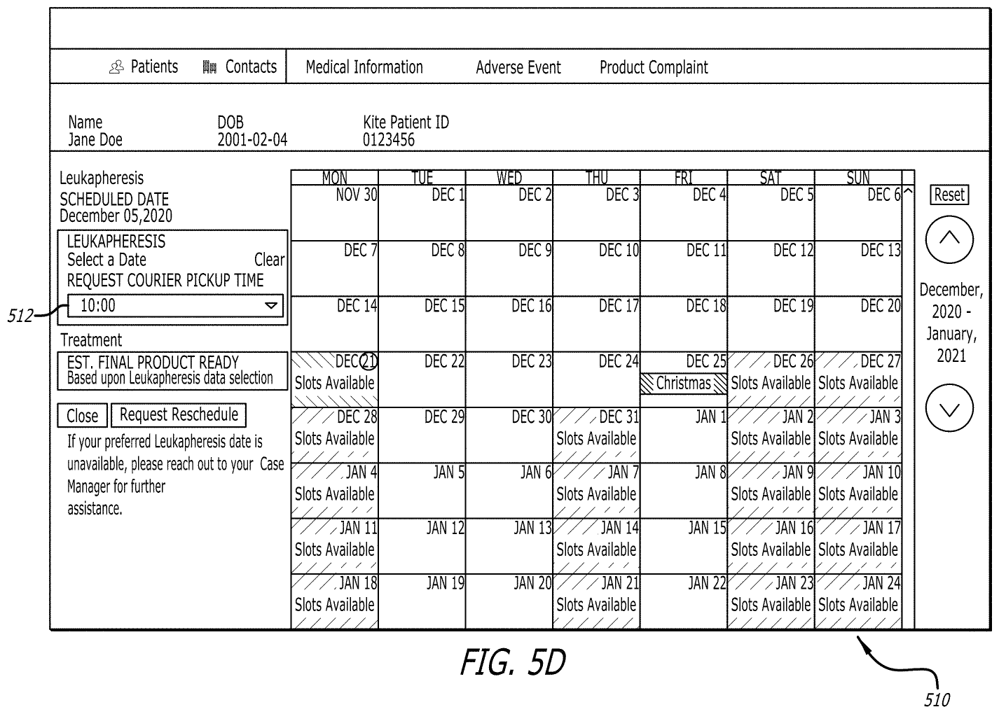

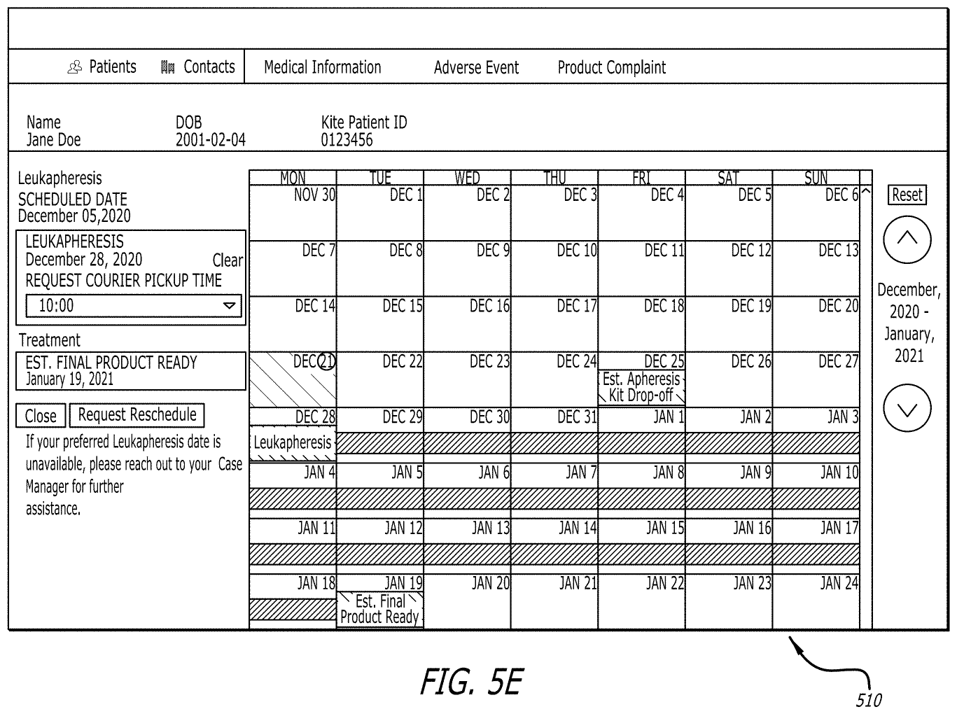

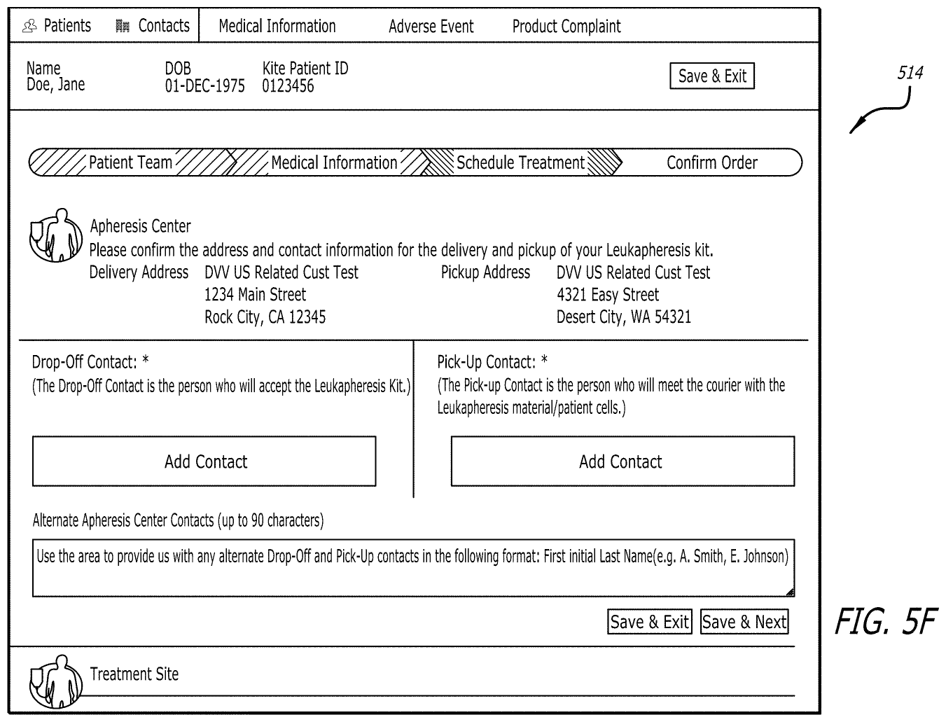

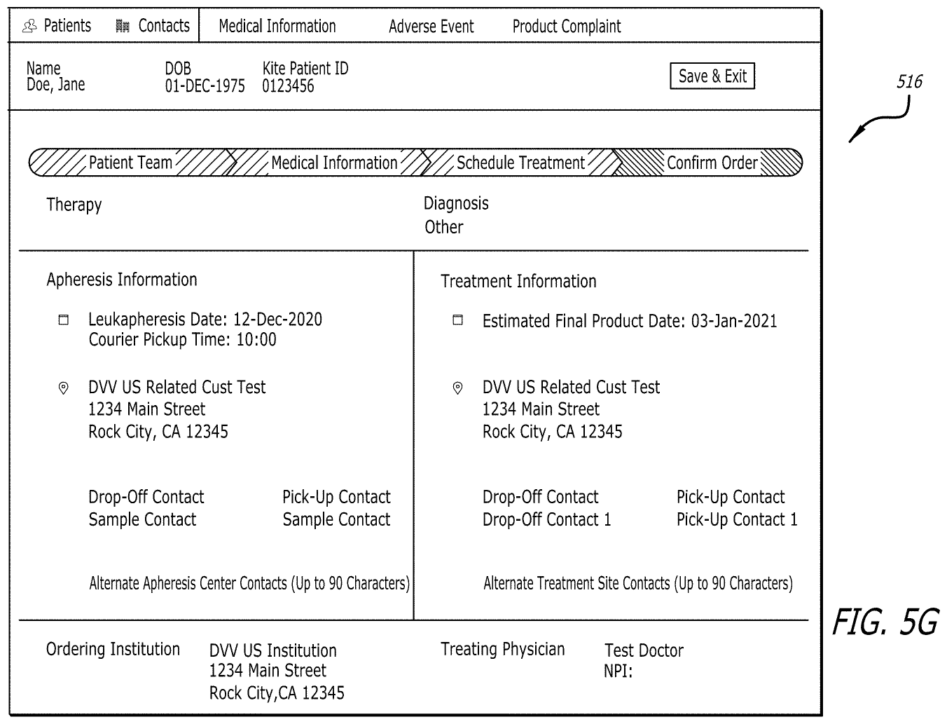

With reference to FIGS. 5A-5G, another embodiment of exemplary screenshots generated by the user interface module 108a that enable confirmation of these sites and scheduling of the appointment is shown. In this embodiment, the scheduling module 114 is built into the patient enrollment platform. This embodiment of the scheduling module 114 shows real time available slots (date and time) for leukapheresis scheduling and manufacturing slot management. In certain embodiments, manufacturing slot management involves the use of system 100 to ensure 1) true available slot capacity at Kite manufacturing facilities are visible and transparent to users, 2) the ability to reserve such slots to be allocated for the use of patient cell manufacturing, and 3) the ability to release slots should they no longer need to be dedicated to their original allocation. The scheduling module 114 may also automatically schedule an apheresis kit drop-off at the treatment center and estimates a final product date. Furthermore, in one embodiment, the scheduling module 114 allows various users, including case managers and health care professionals to request rescheduling and cancellation of patient dates. The scheduling module 114 of this embodiment improves efficiencies and patient experience. In one embodiment, the scheduling module 114 is integrated into the server computing device 106, and in another embodiment, the scheduling module may be incorporated into one of modules 108a, 108b, or 108c.

In one embodiment, various users are able to access the scheduling module, including manufacturing members, health care practitioners, including physicians, case managers, and system administrators. Manufacturing members may access system 100 and the scheduling module 114 to manage reservations and bookings within site and modify capacity (e.g. mark slots for training, maintenance, etc.) as needed to accommodate demand. Also, manufacturing members may access the system 100 to oversee overall manufacturing capacity usage and manage long-term capacity to accommodate demand. The system 100 enables the manufacturing members to establish how manufacturing site capacity is allocated to each day of the week based on treatment site location, the final product, and manufacturing type for each specific immunotherapy procedure. This give the manufacturer or the system administrator the ability to set the total slot capacity limits for each manufacturing site by day such that users of system 100 are prevented from scheduling a higher number of available slots for procedures than the total slot capacity limit on an individual day for the specific manufacturing site.

Health professionals, including physicians may access the system 100 and the scheduling module 114 to view and select Leukapheresis date and courier pick-up time, reschedule, and cancel previously selected dates and times as discussed more below. This may be done during the patient enrollment process.

Case managers may access the system 100 and the scheduling module 114 to view Leukapheresis dates of their patients, courier pick-up times, and estimated final product ready dates requested through patient enrollment. Also, case managers may access the system 100 to receive support requests to approve reschedule and cancellation requests initiated by health care providers. In some embodiments, case managers may enter the system 100 to initiate a cell order request for additional final product from a patient case.

Also, system administrators may access the system 100 and scheduling module 114 to manage overall system rules configurations for the immunotherapy process. In one embodiment, patient users do not have direct access to the system 100. In other embodiments, patient users may be able to view the calendar without making changes or being able to see other patient information.

FIG. 5A is an exemplary screenshot of a system portal page 500 for a patient enrolled in the immunotherapy procedure. A user, such as a physician or health care professional at an Authorized Treatment Center (ATC), ma log into the system 100 and view this screenshot from one of the client computing devices 102a-102d connected to the network 104. As shown in FIG. 5A, the physician or health care professional may view information stored within the database 110 or other memory, including name, ID, type of immunotherapy, treating physician, diagnosis for active and completed patients. In this embodiment, active patients are patients who are currently in the process of being enrolled as patients (e.g. registered status), or who are enrolled and their cells are under the manufacturing process. Completed patients are patients who have received their CAR T cell therapy, and this information is stored for a historical perspective. The page 500 also includes a row of tabs 502 for navigating the system. The tabs may direct a user to other pages that include information patients, contacts, medical information, adverse event, product complaint, request assistance, acknowledgement and notice, and check availability.

By clicking on the “Check Availability” tab on page 500, the user will be directed to an availability calendar page or window 504. An exemplary screenshot of availability calendar window 504 is shown in FIG. 5B. In one embodiment, the availability calendar window 504 displays a complete month on the user interface. In other embodiments, the calendar window 504 may display two or more months on the user interface that the user may scroll through. The calendar window 504 displays current manufacturing slots that are available as well as which patients at the treatment center have been scheduled for the immunotherapy procedure. In one embodiment, the calendar shows dates patients are scheduled for leukapheresis. The available slots and taken slots for leukapheresis are shown on the calendar and updated in real-time. In one embodiment the availability calendar window 504 is updated in real time to all client computing devices 102 in communication with the network. Alerts may be sent to client computing devices 102 as soon as any changes occur to the availability calendar, such as when a leukapheresis slot is filled or becomes available.

In certain embodiments, the availability calendar window 504 may also show holidays, operating hours of the treatment center, physician availability, and the like. Patient names on the availability calendar are also linked to the network, such that a user may click on the name of a patient to view specific patient information. Users may also view the availability calendar with or without having to enroll a patient, which may help in immunotherapy planning. This may allow certain users, such a members of the manufacturing facility or health care providers plan their upcoming schedules.

If a patient if enrolling in an immunotherapy procedure, a date for leukapheresis may be selected on the availability window 504 by clicking on a slot available link. In order to schedule a date for leukapheresis, first, a treating physician would need to have prescribed a CAR T therapy for a patient in order for the enroller (the user of this portal) to proceed with scheduling the appointment. For eligible patients, schedule coordination with the patient and hospital staff would be required concurrent to scheduling this leukapheresis procedure in this calendar of the system 100.

Still referring to FIG. 5B, the user may select a product drop down menu 506 to specify which treatment is shown on the calendar 504. For instance, there are a variety of immunotherapies specific for each patient and by selecting one therapy, only available and filled slots for patients receiving the specific therapy will be shown on the calendar. This may be beneficial to members of a manufacturing facility to view how many upcoming orders for one product are upcoming. In one embodiment, the up-front view of the calendar 504 without needing to enroll a patient may be helpful to all users who have access to the past and future scheduled procedures. By viewing calendar 504, staff at treatment centers will also be able to view where their patients have been scheduled already, as well as see what manufacturing slots are available in real time.

Referring now to FIG. 5C, there is shown an exemplary screen shot of a patient record window 508 having a scheduled leukapheresis or apheresis date and an estimated final product date. Along with the dates, the location of the apheresis location, manufacturing location, and final product drop off location for infusion back into the patient may also be provided in one embodiment. Also shown in one embodiment is the manufacturing status that provides where in the overall process of cell manufacturing the patient's cells are. Any delays in the manufacturing process that may affect the final product delivery date may be shown here. In certain embodiments the date for final product is an estimate date that may change. The final product date is an extrapolation based on the planned apheresis date. In one embodiment, this date is typically 18 calendar days after the apheresis procedure takes place. In other embodiments, the date of the final product being ready may depend on the manufacturing location compared to the location of the treatment and other external factors including weather and other elements that may affect transportation of the final product. The final product date is automatically generated by the system 100, in one embodiment. Also, the user may manually enter the final product date into the system or override the automatically generated date.

FIG. 5D depicts an exemplary screen shot of a single patient calendar or rescheduling calendar 510 where a user can select a new date for leukapheresis or apheresis. As shown, this webpage or window is specific to a single patient. The rescheduling calendar 510 (specific to a single patient) may be another view of the availability calendar 504 (shows all patients scheduled), but specific to a single patient. In one embodiment, once an available slot for leukapheresis is selected for the patient, the previous scheduled date is automatically cancelled if it was not already cancelled manually by the user. Furthermore, once a date for the patient is scheduled, the user may also select a requested courier pickup time in drop down menu 512. In one embodiment a case manager or health care provider will select the request courier pickup time. The courier will pick up the apheresis collected from the patient and transport it to the manufacturing facility to engineer the patient's cells. This ensures a fast transition from collecting cells from a patient to transporting them to the manufacturing facility. The available slots and taken slots for leukapheresis are shown on the calendar and updated in real-time. In one embodiment the calendar window 510 is updated in real time to all client computing devices 102 in communication with the network. Alerts may be sent to client computing devices 102 as soon as any changes occur to the availability calendar, such as when a leukapheresis slot is filled or becomes available.

Once a date for the patient is selected on calendar 510 (or calendar 504) the calendar window 510 may display the estimated final product date as shown in the exemplary screen shot of FIG. 5E. The estimated final product date is the date the final product of engineered cells from the patient may be delivered to the hospital for infusion into the patient. Again, since time is of the essence for immunotherapy patients, it is important to know the delivery date of the final product so that the patient can prepare for infusion of the final product with little to no delay. In this embodiment, once the date for the patient is selected, a date for the apheresis kit drop-off will also be set and shown on the calendar 510. The date for the kit drop-off can be any time before the scheduled leukapheresis. The apheresis kit will include all materials, such as bags, and shippers, for shipping the apheresis once collected. The system 100 can automatically send an alert to the manufacturing facility for them to prepare and ship the apheresis kit to the treatment facility performing the leukapheresis or apheresis. In one embodiment the case manager will confirm with the site and schedule the apheresis kit for drop-off. In some embodiments, the user interface module 108a communicates with a remote computing device of the manufacturing facility, in conjunction with the database 110, to coordinate scheduling of the biological material modification to ensure the most efficient processing schedule so that the modified material is returned quickly back to the patient. This helps with ensuring the collection of cells from a patient occurs with little delay. Once the date has been selected and the manufacturing, kit drop-off and final product drop off dates are generated, the system in one embodiment may require an administrator or case manager to review all dates to ensure accuracy and follow up with the patient.

In certain embodiments, a user may return to calendar 504 or calendar 510 in order to cancel all selected dates for a patient. In another embodiment, the user is allowed to return to calendar 510 in order to schedule, reschedule, or cancel an appointment date. In this embodiment, calendar 504 is for viewing only. The system will update all calendars in real time so that any user viewing the calendar will immediately see any cancellations or changes made to the calendar. In one embodiment, all or certain users of the system 100 will receive an alert once a patient cancels a scheduled date.

In one embodiment shown in FIG. 5F, which depicts an exemplary screenshot of a contact window 514, the user is allowed to specify drop-off and pickup contacts at the treatment centers. The drop-off contact may receive the apheresis drop-off kit and the pickup contact is the person who will meet the courier with the apheresis material/patient cells. Additional backup contacts can also be added. In certain embodiments, the calendar 504 or 510 will be display below the treatment site contacts.

A final confirmation page 516 of one embodiment is shown in FIG. 5G. On this page or window 516 is a summary of the entire immunotherapy procedure, including the type of therapy, the leukapheresis data, courier pickup time, location of the treatment facility, drop-off and pick-up contacts, treatment information, final product date and location of the treatment facility that will perform the infusion.

Turning back to FIG. 2, once the cell order process is complete as described above, a process 206 is initiated to perform the biological material extraction procedure at the extraction site, ship the extracted material to the manufacturing facility for modification, and send the modified material back to a delivery site for infusion back into the patient's bloodstream. First, the patient arrives at the material extraction site and a procedure (e.g., a leukapheresis procedure) is performed (206a) on a sample of the patient's blood to collect T cells from the sample. When the procedure is performed, a client computing device (e.g., device 102b) at the extraction site communicates with the event tracking module 108b of server computing device 106 to transmit a tracking event to the module 108b that corresponds to performance of the procedure. For example, a clinician at client computing device 102b may submit the tracking event by entering information into a user interface. In another example, the client computing device 102b may automatically transmit the tracking event to the module 108b (e.g., via API) when information about the procedure is captured by the client computing device 102b (e.g., scanning a barcode).

The tracking event may comprise the patient-specific identifier, a timestamp, an event ID (e.g., that indicates a material extraction procedure was performed), and other information relevant to the process (e.g., cell order lot number, sales order number, site location, etc.). The event tracking module 108b stores the tracking event in database 110 based upon the information received from the client computing device 102b. Because this is the first step in the biological material extraction and modification process, the event tracking module 108b notifies the chain of custody module 108c of receipt of the tracking event. The chain of custody module 108c generates a chain of custody data structure (e.g., in database 110) that incorporates the tracking event (and each subsequent tracking event described herein) in an ordered sequence that enables the patient, the physician, the manufacturer, and other parties to understand the precise status of the biological material and to ensure that the biological material is accounted for at all times in avoidance of loss or mishandling. In an example, the chain of custody data structure may be a linked list that connects each of the tracking events together in a sequential manner according to, e.g., timestamp of the tracking event.

Next, the collected T cells are transferred (206b) to a container (e.g., a tube, vial, or other type of biological material carrier) and another tracking event is captured and transmitted to the event tracking module 108b for integration into the chain of custody data structure described above. Then, the container is labeled (206c) with the patient-specific identifier, and another tracking event is captured and transmitted to the event tracking module 108b for communication with the chain of custody module 108c to integrate into the chain of custody data structure. For example, the container that houses the collected T cells is labeled with a barcode comprising the patient-specific identifier, which is then scanned at the extraction site—indicating that the collected T cells are ready for shipment to the manufacturing facility. Upon scanning the barcode, the client computing device 102b generates the tracking event and transmits the event to the event tracking module 108b.

Then, the extraction site transmits (206d) the collected T cells to the manufacturing facility, which performs the procedure to generate the transfected T cells. Both when the collected T cells are shipped to the manufacturing facility and when the collected T cells are received at the manufacturing facility, one or more of the devices used to record the shipment and receipt of the T cells communicate with the event tracking module 108b to transmit a tracking event associated with the particular activity for communication with the chain of custody module 108c to integrate into the chain of custody. In this way, the chain of custody module 108c automatically and continuously updates the chain of custody data structure with the latest information, and that information is reflected in one or more screens generated by the user interface module 108a.

The manufacturing facility then creates (206e) transfected T cells from the collected T cells using a cell modification technique, and a client computing device (e.g., device 102c) generates one or more tracking events based upon the particular cell modification technique being used. For example, a cell modification technique may comprise several phases—such as (i) quality assurance of the collected T cells prior to modification, (ii) modification of the T cells; (ii) release testing of the transfected T cells, and (iv) finalization of the transfected T cells for shipment back to the infusion site. For each of these phases, the client computing device 102c captures a tracking event and transmits the tracking event to the event tracking module 108b for integration by the chain of custody module 108c into the chain of custody data structure.

Once the transfected T cells are shipped, the infusion site receives (2061) the transfected T cells and a client computing device (e.g., device 102d) generates a tracking event for transmission to the event tracking module 108b for integration by the chain of custody module 108c into the chain of custody data structure. For example, the client computing device 102d may scan a barcode associated with the shipment and/or the transfected T cells to automatically generate the tracking event and transmit the event to the server computing device 106.

After receipt, the transfected T cells are infused (206g) into the patient's bloodstream, thereby completing the process. At the same time, the client computing device 102d generates a tracking event and transmits the event to the event tracking module 108b for integration by the chain of custody module 108c into the chain of custody data structure.

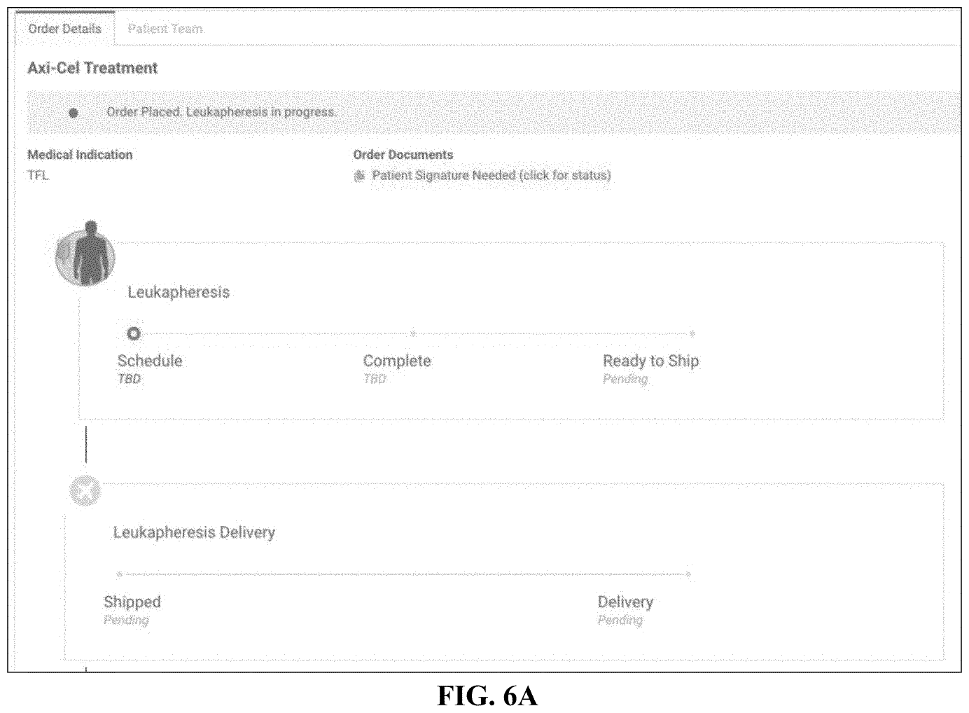

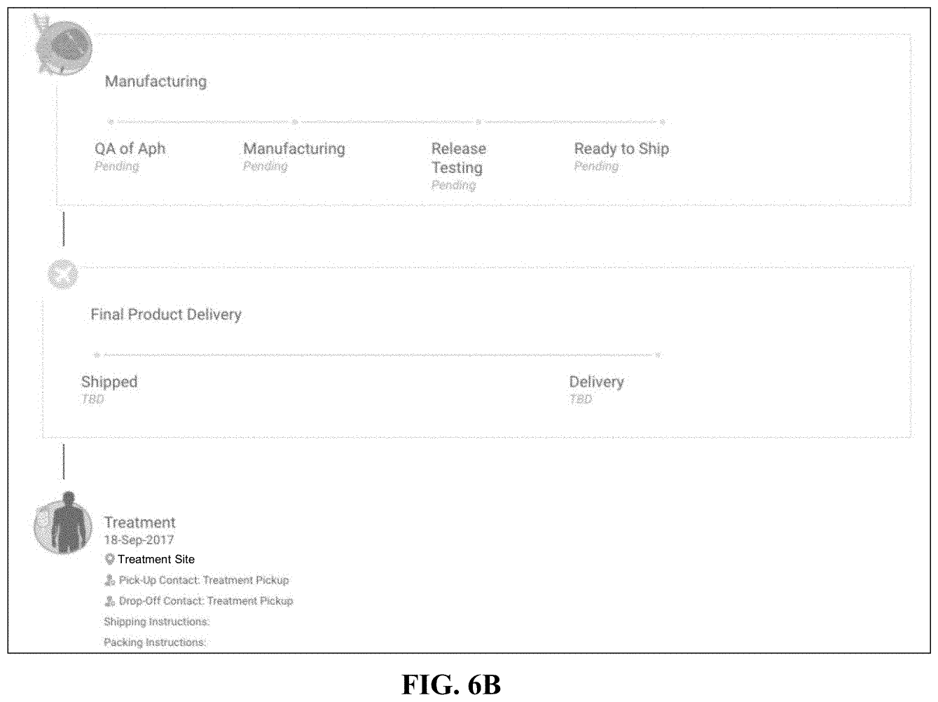

FIGS. 6A and 6B are exemplary screenshots generated by the user interface module 108a to enable the client computing devices 102a-102d to view the chain of custody associated with a particular patient, biological material, and cell modification process. As shown in FIG. 6A, the chain of custody of the biological material during the leukapheresis process (including the steps of scheduling the procedure, completing the procedure, and having the extracted T cells ready for shipment) is captured in a timeline at the top of the screen, where each step of the leukapheresis process is associated with a point on the timeline, and the chain of custody of the biological material during the delivery process (e.g., T cells shipped from extraction site, T cells delivered to manufacturing facility) is captured in a timeline at the bottom of the screen. When the event tracking module 108b and chain of custody module 108c record a tracking event as described above, the user interface module 108a traverses the chain of custody data structure to graphically represent the current status of the chain of custody on screen.

As shown in FIG. 6B, the chain of custody of the biological material during the manufacturing process (including QA, manufacturing, release testing, and finalizing for shipment) is shown in a timeline at the top of the screen, and the chain of custody of the biological material during the final product delivery process (including shipment and delivery to the infusion site) is shown in the middle of the screen. In addition, the treatment details, including the treatment date, are displayed at the bottom of the screen. Also, the chain of custody is constantly associated with the specific patient—thereby ensuring a complete chain of identity between the patient and the biological material during all phases of manufacturing.

The above-described techniques may be implemented in digital and/or analog electronic circuitry, or in computer hardware, firmware, software, or in combinations of them. The implementation may be as a computer program product, i.e., a computer program tangibly embodied in a machine-readable storage device, for execution by, or to control the operation of, a data processing apparatus, e.g., a programmable processor, a computer, and/or multiple computers. A computer program may be written in any form of computer or programming language, including source code, compiled code, interpreted code and/or machine code, and the computer program may be deployed in any form, including as a stand-alone program or as a subroutine, element, or other unit suitable for use in a computing environment. A computer program may be deployed to be executed on one computer or on multiple computers at one or more sites. The computer program may be deployed in a cloud computing environment (e.g., Amazon® AWS, Microsoft® Azure, IBM®).

Method steps may be performed by one or more processors executing a computer program to perform functions of the disclosed system by operating on input data and/or generating output data. Method steps may also be performed by, and an apparatus may be implemented as, special purpose logic circuitry, e.g., a FPGA (field programmable gate array), a FPAA (field-programmable analog array), a CPLD (complex programmable logic device), a PSoC (Programmable System-on-Chip), ASIP (application-specific instruction-set processor), or an ASIC (application-specific integrated circuit), or the like. Subroutines may refer to portions of the stored computer program and/or the processor, and/or the special circuitry that implement one or more functions.

Processors suitable for the execution of a computer program include, by way of example, special purpose microprocessors specifically programmed with instructions executable to perform the methods described herein. Generally, a processor receives instructions and data from a read-only memory or a random-access memory or both. The essential elements of a computer are a processor for executing instructions and one or more memory devices for storing instructions and/or data. Memory devices, such as a cache, may be used to temporarily store data. Memory devices may also be used for long-term data storage. Generally, a computer also includes, or is operatively coupled to receive data from or transfer data to, or both, one or more mass storage devices for storing data, e.g., magnetic, magneto-optical disks, or optical disks. A computer may also be operatively coupled to a communications network in order to receive instructions and/or data from the network and/or to transfer instructions and/or data to the network. Computer-readable storage mediums suitable for embodying computer program instructions and data include all forms of volatile and non-volatile memory, including by way of example semiconductor memory devices, e.g., DRAM, SRAM, EPROM, EEPROM, and flash memory devices; magnetic disks, e.g., internal hard disks or removable disks; magneto-optical disks; and optical disks, e.g., CD, DVD, HD-DVD, and Blu-ray disks. The processor and the memory may be supplemented by and/or incorporated in special purpose logic circuitry.

To provide for interaction with a user, the above described techniques may be implemented on a computing device in communication with a display device, e.g., a CRT (cathode ray tube), plasma, or LCD (liquid crystal display) monitor, a mobile device display or screen, a holographic device and/or projector, for displaying information to the user and a keyboard and a pointing device, e.g., a mouse, a trackball, a touchpad, or a motion sensor, by which the user may provide input to the computer (e.g., interact with a user interface element). Other kinds of devices may be used to provide for interaction with a user as well; for example, feedback provided to the user may be any form of sensory feedback, e.g., visual feedback, auditory feedback, or tactile feedback; and input from the user may be received in any form, including acoustic, speech, and/or tactile input.

The above-described techniques may be implemented in a distributed computing system that includes a back-end component. The back-end component may, for example, be a data server, a middleware component, and/or an application server. The above described techniques may be implemented in a distributed computing system that includes a front-end component. The front-end component may, for example, be a client computer having a graphical user interface, a Web browser through which a user may interact with an example implementation, and/or other graphical user interfaces for a transmitting device. The above described techniques may be implemented in a distributed computing system that includes any combination of such back-end, middleware, or front-end components.

The components of the computing system may be interconnected by transmission medium, which may include any form or medium of digital or analog data communication (e.g., a communication network). Transmission medium may include one or more packet-based networks and/or one or more circuit-based networks in any configuration. Packet-based networks may include, for example, the Internet, a carrier internet protocol (IP) network (e.g., local area network (LAN), wide area network (WAN), campus area network (CAN), metropolitan area network (MAN), home area network (HAN)), a private IP network, an IP private branch exchange (IPBX), a wireless network (e.g., radio access network (RAN), Bluetooth, near field communications (NFC) network, Wi-Fi, WiMAX, general packet radio service (GPRS) network, HiperLAN), and/or other packet-based networks. Circuit-based networks may include, for example, the public switched telephone network (PSTN), a legacy private branch exchange (PBX), a wireless network (e.g., RAN, code-division multiple access (CDMA) network, time division multiple access (TDMA) network, global system for mobile communications (GSM) network), and/or other circuit-based networks.

Information transfer over transmission medium may be based on one or more communication protocols. Communication protocols may include, for example, Ethernet protocol, Internet Protocol (IP), Voice over IP (VOIP), a Peer-to-Peer (P2P) protocol, Hypertext Transfer Protocol (HTTP), Session Initiation Protocol (SIP), H.323, Media Gateway Control Protocol (MGCP), Signaling System #7 (SS7), a Global System for Mobile Communications (GSM) protocol, a Push-to-Talk (PTT) protocol, a PTT over Cellular (POC) protocol, Universal Mobile Telecommunications System (UMTS), 3GPP Long Term Evolution (LTE) and/or other communication protocols.

Devices of the computing system may include, for example, a computer, a computer with a browser device, a telephone, an IP phone, a mobile device (e.g., cellular phone, personal digital assistant (PDA) device, smart phone, tablet, laptop computer, electronic mail device), and/or other communication devices. The browser device includes, for example, a computer (e.g., desktop computer and/or laptop computer) with a World Wide Web browser (e.g., Chrome™ from Google, Inc., Microsoft® Internet Explorer® available from Microsoft Corporation, and/or Mozilla® Firefox available from Mozilla Corporation). Mobile computing device include, for example, a Blackberry® from Research in Motion, an iPhone® from Apple Corporation, and/or an Android™-based device. IP phones include, for example, a Cisco® Unified IP Phone 7985G and/or a Cisco® Unified Wireless Phone 7920 available from Cisco Systems, Inc.

One skilled in the art will realize the subject matter may be embodied in other specific forms without departing from the spirit or essential characteristics thereof. The foregoing embodiments are therefore to be considered in all respects illustrative rather than limiting of the subject matter described herein.

What is claimed is:

1. A method of scheduling a patient-specific immunotherapy procedure, the method comprising:
receiving a request on a scheduling module to schedule a leukapheresis appointment for an available date and time displayed on a calendar stored in a database that is in communication with the scheduling module;
updating the calendar in real time by the scheduling module;
automatically generating a final product date by the scheduling module to estimate when the final product of engineered cells from the patient will be available for infusion into the patient; and
receiving manufacturing process updates for the final product of engineered cells from the patient on the scheduling module and updating a status of the manufacturing process.

2. The method of claim 1, further comprising automatically scheduling by the scheduling module an apheresis kit drop-off time before the scheduled leukapheresis appointment, and updating the calendar in real time to include the drop off time for the apheresis kit.

3. The method of claim 2, further comprising automatically sending an alert from the scheduling module to a remote facility with the scheduled drop-off time and a desired location for the apheresis kit, wherein the remote facility will deliver the apheresis kit at the scheduled drop-off time to the desired location.

4. The method of claim 1, further comprising receiving a request on the scheduling module to schedule a courier pick-up time for cells collected from the patient during the scheduled leukapheresis appointment.

5. The method of claim 1, wherein updating the calendar in real time displays available date and time slots for leukapheresis appointments and the booked date and time slots for leukapheresis appointments.

6. The method of claim 1, wherein receiving manufacturing process updates on the scheduling module in real time.

7. The method of claim 1, further comprising receiving a request on the scheduling module to reschedule the leukapheresis appointment for another available date and time displayed on the calendar, and updating the calendar in real time by the scheduling module.

8. The method of claim 1, further comprising receiving a request on the scheduling module to cancel the scheduled leukapheresis appointment and updating the calendar in real time by the scheduling module.

9. The method of claim 1, further comprising receiving a number of available leukapheresis appointments for each day on the calendar on the scheduling module.

10. A system for scheduling a patient-specific immunotherapy procedure, the system comprising:
a scheduling module that provides a calendar with available dates and times for scheduling a leukapheresis appointment;
a network in communication with the scheduling module; and
a plurality of client computing devices in communication with the scheduling module via the network,
wherein one of the client computing devices sends a request to the scheduling module to schedule an available leukapheresis appointment date and time for the patient,
wherein the scheduling module receives the request to schedule the leukapheresis appointment for the patient and updates the calendar in real time that can be accessed by each of the plurality of client computing devices,
wherein the scheduling module automatically generates a final product date based on the scheduled leukapheresis appointment date and time, the final product date estimates when the final product of engineered cells from the patient will be available for infusion into the patient, and the scheduling module updates the calendar with the final product date.

11. The system of claim 10, wherein one of the client computing devices sends manufacturing process updates for the final product of engineered cells from the patient to the server computing device via the network.

12. The system of claim 10, further comprising a database in communication with the scheduling module to store the updated calendar.

13. The system of claim 10, wherein the scheduling module automatically schedules an apheresis kit drop-off time before the scheduled leukapheresis appointment and updates the calendar to include the drop off time for the apheresis kit.

14. The system of claim 13, wherein scheduling module automatically sends an alert to a remote facility with the scheduled drop-off time and a desired location for the apheresis kit, wherein the remote facility will deliver the apheresis kit at the scheduled drop-off time to the desired location.

15. The system of claim 10, wherein one of the computing devices sends a request to the scheduling module via the network to schedule a courier pick-up time for cells collected from the patient during the scheduled leukapheresis appointment.

16. The system of claim 10, wherein the scheduling module updates the calendar in real time to display available date and time slots for leukapheresis appointments and booked date and time slots for leukapheresis appointments.

17. The system of claim 10, wherein one of the computing devices requests the scheduling module to reschedule the leukapheresis appointment for another available date and time displayed on the calendar.

18. The system of claim 17, wherein the scheduling module updates the calendar with the rescheduled leukapheresis appointment in real time.

19. The system of claim 10, wherein one of the computing devices requests the scheduling module to cancel the scheduled leukapheresis appointment.

20. The system of claim 19, wherein the scheduling module updates the calendar with the canceled leukapheresis appointment in real time.

21. The system of claim 10, wherein one of the computing devices sends to the scheduling module via the network a number of available leukapheresis appointments for each day on the calendar.
